(19) United States
(12) Patent Application Publication
(10) Pub. No.: US 2026/0102449 A1
(43) Pub. Date: Apr. 16, 2026

(54) NUTRITIONAL SUPPLEMENT, DRINK MIX, AND DRINK MADE THEREFROM

(71) Applicant: FONTEM SANCTUS FRUCTUS, INC., Fruitland, UT (US)

(72) Inventor: Michael KRAVET

(21) Appl. No.: 18/936,088

(22) Filed: Nov. 4, 2024

Related U.S. Application Data
(63) Continuation-in-part of application No. 18/917,845, filed on Oct. 16, 2024.

(51) Int. Cl. A61K 36/07; A23L 2/66; A61K 9/00; A61K 31/198; A61K 31/522; A61K 36/068; A61K 36/074; A61K 36/185; A61K 36/54; A61K 36/898; A61K 38/39; A61P 1/00; A61P 17/02; A61P 19/02; A61P 25/28

(52) U.S. Cl. CPC A61K 36/07; A23L 2/66; A61K 9/0095; A61K 31/198; A61K 31/522; A61K 36/068; A61K 36/074; A61K 36/54; A61K 36/5777; A61K 36/898; A61K 38/39; A61P 1/00; A61P 17/02; A61P 19/02; A61P 25/28

(57) ABSTRACT

A nutritional supplement composition formulated to provide improved mental focus and clarity, improved bowel function, and deceased pain associated with arthritis. The composition includes cacao, collagen protein, cinnamon, vanilla, mushroom/fungus derived components (some or all of reishi, chaga, turkey tail, lion's mane, and cordyceps), and natural salt. Because cacao is the primary or sole source of stimulant components in the composition, the amount of caffeine is significantly lower than typical caffeinated energy drinks and products. The ratio of theobromine to caffeine can be at least about 5:1 and as high as about 15:1 (or more). The collagen protein promotes improved joint function and acts synergistically with the other components to improve mood and mental clarity.

### CROSS-REFERENCE TO RELATED APPLICATIONS

This application is a continuation-in-part of U.S. patent application Ser. No. 18/917,845, filed Oct. 16, 2024, which is incorporated by reference in its entirety.

### BACKGROUND

Caffeinated beverages, such as coffee, tea, and energy drinks are used by consumers to promote wakefulness, maintain alertness, and enhance mood and cognition. Typical gas station energy drinks include common ingredients such as caffeine and sugar. Some may also include vitamins, electrolytes, or amino acids.

Despite widespread use, there are several health concerns surrounding consumption of highly caffeinated beverages, particularly negative effects on the cardiovascular system and the tendency to raise stress levels. Highly caffeinated energy drinks have been shown to increase heart rate, increase blood pressure, and even thicken the blood. Highly caffeinated energy drinks may also affect the brain in undesirable ways, such as by raising stress levels and inducing anxiety, insomnia, gastrointestinal irritation, muscle twitching, and periods of prolonged restlessness.

Highly caffeinated energy drinks are also associated with high levels of fatigue, irritability, and sub-optimal cognition following the initial boost after consumption. This energy “crash” can be long lasting and unpleasant. As the stimulating effects wear off, levels of adrenaline, dopamine, and acetylcholine begin to drop, and blood sugar levels may swing erratically, often leaving the user feeling more fatigued, irritable, and unfocused than before consuming the energy drink. In the case of caffeine in particular, the user's adenosine levels may continue to rise after dosing. When caffeine is no longer available to block adenosine receptors, the rapid flood of built-up adenosine to the receptors can contribute to the experienced crash.

To date, common energy drinks have generally failed to improve the overall quality of life and well-being of users. Rather, such energy drinks provide a short-lived boost in desired alertness but with a subsequent crash that often tends to be so uncomfortable as to negate the benefits and place consumption of energy drinks as a net negative experience. When used to stay awake while driving, common energy drinks can cause high levels of stress and possibly contribute to “road rage”. For long haul drives, the mental and/or physical “crash” by the user is potentially harmful or even fatal if it leads to a highway crash.

There are number of coffee substitutes that purportedly contain less caffeine while providing improved mental clarity. One is Mudwtr, which is based on masala black chai tea, cacao, mushrooms, turmeric, and other substances. While Mudwtr may provide the purported benefits, one of the problems is poor dispersion, requiring frequent stirring to maintain the components in suspension rather than sinking to the bottom of the cup.

Accordingly, there is an ongoing need for compositions that are capable of promoting a natural and sustained feeling of alertness without jitteriness, while increasing concentration, enhancing mood, and/or enhancing cognition without the typical energy crash and without damaging the long-term physiological or mental health of the user.

### SUMMARY

Disclosed herein are nutritional supplement compositions formulated to provide mental clarity, including treating the effects of attention deficit disorder (ADD), without the jitteriness associated with typical highly caffeinated beverages. The nutritional supplement compositions have also been found to provide additional unexpected health benefits including, but not limited to, general reduction in pain, including reduction in pain associated with arthritis, increased youthfulness, improved gastrointestinal function, including improved bowel function, increased immunity to sickness, faster healing of wounds, and reduced desire to consume sugar and other high glycemic index carbohydrates.

In some embodiments, the nutritional supplement compositions are in the form of an additive, such as a power, that can be dispersed in a liquid, such as warm to hot water to make a nutritional beverage product, or mixed with a solid to make an edible nutritional food product. The nutritional supplement compositions generally comprise most or all of the following components in relative order of prominence: cacao, collagen protein, cinnamon powder, vanilla powder, reishi mushroom, chaga fungus, turkey tail mushroom, lion's mane mushroom, cordyceps fungus (collectively “fungus components”, “mushroom components”, or “mushroom/fungus components”), and natural salt, wherein the mushroom/fungus components can be in the form of a powder, such as ground fruiting bodies and/or mycelium, and extracts made therefrom.

The foregoing components, when included in specific amounts and ratios in the nutritional supplement compositions and products made therewith, have been found to provide a surprising and unexpected range of benefits, such as the aforementioned. An important aspect of the invention is the inclusion of relatively high amounts of cacao, collagen protein, cinnamon, and vanilla, together with modest but significant amounts of the remaining components, including modest but significant amounts of the mushroom/fungus components (reishi, chaga, turkey tail, lion's mane, and cordyceps) and natural salt.

More importantly, because cacao is typically the primary or sole source of stimulant components in the nutritional supplement composition, the amount of caffeine is typically lower than typical caffeinated energy drinks and products. More importantly, because cacao has a very high ratio of theobromine to caffeine, the generally harsh effects of caffeine are offset by the moderating effects of the theobromine. For example, while caffeine is a vasoconstrictor, which can increase blood pressure and a feeling of stress and anxiety, theobromine is a vasodilator that relaxes blood vessels, producing a feeling of calm, unstressed mental clarity and focus. The ratio of theobromine to caffeine is reportedly at least about 6:1 and can be as high as about 15:1, depending on the source and manner of processing the raw cacao beans. Cacao is also high in iron, magnesium, copper, potassium, selenium, zinc, and phosphorus. Cacao acts as an antioxidant, increases HDL production, is high in flavanols, which improve skin health, and improves cognitive function.

The inclusion of relative high amounts of collagen protein (e.g., beef collagen) in the nutritional supplement composition has also been found to provide substantial benefits when included with the other components. For example, collagen protein is the major protein component of soft joint tissues, including cartilage. When combined with the other components, collagen protein has been found to promote healthier joints and to relieve joint point, including pain associated with arthritis. Another benefit of collagen protein, one that has not been readily recognized, is its positive affect on mood and mental clarity, particularly when combined with cacao and the other components. Other benefits include bone loss prevention, increased skin and joint elasticity, anti-inflammatory, anti-aging, and healthy bile production. The collagen protein also appears to maintain good dispersion the other components of the nutritional supplement composition when mixed into water or other liquid, as the other components do not readily sink to the bottom of the cup over time as occurs with coffee substitutes that include mushroom components, cacao, etc.

In some embodiments, the nutritional supplement composition in powder form can be combined with warm or hot water to form a beverage that can be consumed one or more times daily. The drink can be consumed as is or flavored by adding milk and/or honey.

Additional features and advantages will be set forth in part in the description that follows, and in part will be obvious from the description, or may be learned by practice of the embodiments disclosed herein. It is to be understood that both the foregoing brief summary and the following detailed description are exemplary and not restrictive of the embodiments disclosed herein or as claimed.

### DETAILED DESCRIPTION

### I. Introduction and Definitions

Disclosed are nutritional supplement compositions formulated to provide mental clarity, including treating the effects of attention deficit disorder (ADD), without the jitteriness associated with typical highly caffeinated beverages. The compositions have also been found to provide additional unexpected health benefits including, but not limited to, boosting energy, generally reducing pain, including reducing pain associated with arthritis, increasing youthfulness, improving gastrointestinal function, including improving bowel function, increasing immunity to sickness, increasing rate of wound healing, improving skin health and elasticity, increasing HDL production, providing anti-inflammatory effects, providing anti-oxidant activity, providing anti-viral activity, improves cognitive function, providing probiotics, improving mood, reducing depression and anxiety, providing neuroprotection, boosting immunity, regulating blood pressure, regulating blood sugar, anti-aging, hormone regulation, reducing adrenaline spikes, maintaining pH, improving melatonin production, improving metabolism, supporting thyroid function, reducing the desire to consume sugar and other high glycemic index carbohydrates.

In some embodiments, the nutritional supplement compositions are in the form of power that is dispersible in a liquid, such as warm to hot water, to form a nutritional beverage, or to a sold to form a nutritional food product. The nutritional supplement compositions generally comprise most or all of the following components in relative order of prominence: cacao, collagen protein, cinnamon powder, vanilla powder, reishi mushroom, chaga fungus, turkey tail mushroom, lion's mane mushroom, cordyceps fungus (collectively “fungus components”, “mushroom components”, or “mushroom/fungus components”), and natural salt. The mushroom/fungus components can be in the form of a powder, such as ground fruiting bodies and/or mycelium, and extracts made therefrom. In some embodiments, the nutritional supplement composition can be consumed as a beverage or food produce as dosage form one or more times daily. The beverage produce can be consumed as is or flavored by adding milk and/or honey. Coconut oil and/or MCT oil can be added to provide a source of fat-derived calories.

As used herein, the term “crash” or “crash effect” refers to the state a user may experience following consumption of a stimulant such as caffeine. A crash is characterized by unpleasant effects such as extreme tiredness, difficulty concentrating, irritability, diminished judgment, and/or poor mood. As the stimulant wears off, neurotransmitter and/or hormone (e.g., dopamine, acetylcholine, adrenaline) levels may drop relatively sharply, and blood sugar level swings may become larger than normal. Insulin spikes may also exacerbate the crash. In the case of caffeine in particular, adenosine levels may continue to rise after dosing. When caffeine is no longer available to block adenosine receptors, the rapid flood of built-up adenosine to the receptors can contribute to the crash.

The individual components of the nutritional supplement compositions beneficially and synergistically interact together to enhance the beneficial effects of the nutritional supplement composition and provide a more natural feeling mental clarity boosting effect. For example, the strong vasodilating effects of theobromine counteract the vasoconstriction effects of caffeine, which essentially eliminates the usual physical sensations of heart palpitations and stress caused by caffeine. In addition, the total amount of caffeine is relatively so, which further reduces the typical agitation and feeling of anxiety caused by consumption of high amounts of caffeine. Theobromine can functions to improve the pharmacokinetic distribution of the other components. In other words, the vasodilating effects of theobromine function to increase blood flow to peripheral tissues of the body so that the other components of the composition can more effectively move from the digestive system to their intended targets (e.g., brain, muscle tissue, connective tissue, and joint tissue).

As used herein, the term “energy level,” “experienced energy level,” or “subjective energy level” refers to the level of wakefulness and/or focus experienced by the user. They can be both mental and physical in nature. While these may be relatively subjective properties, they are readily understood by users and are amenable to measurement through appropriate questioning and/or surveying. For example, as with various clinical mood or pain rating measurements, a user may describe energy level, mood, and/or pain on a scale (e.g., 1 to 10 or 1 to 100).

The term “administration” or “administering” is used herein to describe the process in which a nutritional supplement composition and products made therewith is taken by or delivered to a subject. The nutritional supplement composition will typically be administered by oral ingestion as a beverage or food product.

The term “dosage form” refers to the structural form in which the nutritional supplement composition is provided and/or the vehicle by which the nutritional supplement composition is delivered to the user. Example dosage forms include, but are not limited to, tablets, capsules, powders, gels, food products, food additives, beverages/drinks (e.g., in cans, bottles, cartons, pouches, squeeze containers, and the like), beverage additives, solid food products food supplements, sprays, and suppositories. Preferred embodiments of the nutritional supplement composition are formulated as liquids (e.g., beverages) or solids (e.g., food products) for oral ingestion.

The term “unit dose” refers to a dosage form that is configured to deliver a specified quantity or dose of the nutritional supplement composition or component thereof. Dosage forms may be configured to provide a full unit dose or fraction thereof (e.g., ½, ⅓, or ¼ of a unit dose).

Another dosage form that can be used to provide a unit dose of the nutritional supplement composition or component thereof is a “unit dose measuring device”, such as a cup, scoop, syringe, dropper, spoon, spatula, or colonic irrigation device, which is configured to hold therein a measured quantity of the composition equaling a full unit dose or fraction thereof (e.g., ½, ⅓, or ¼ of a unit dose). An example use of such measuring device(s) is in kit together with a bulk container.

For example, a “bulk container”, such as a carton, box, can, jar, bag, pouch, bottle, jug, or keg, containing several unit doses of the nutritional supplement composition (e.g., 5-250 or 10-150 unit doses) can be provided to a user together with a unit dose measuring device that is configured to provide a unit dose, or fraction thereof, of the composition or component thereof.

A “kit” for use in providing a nutritional supplement composition as disclosed herein in bulk form, while providing unit doses of the composition, may comprise a bulk container holding therein a quantity of composition and a unit dose measuring device configured to provide a unit dose, or fraction thereof, of composition or component thereof. One or more unit dose measuring devices may be positioned inside the bulk container at the time of sale, attached to the outside of the bulk container, prepackaged with the bulk container within a larger package, or provided by the seller or manufacture for use with one or multiple bulk containers. A bulk container may contain liquid or solid forms of the composition.

The kit may include instructions regarding the size of the unit dose, or fraction thereof, and the manner and frequency of administration. The instructions may be provided on the bulk container, prepackaged with the bulk container, placed on packaging material sold with the bulk container, or otherwise provided by the seller or manufacturer (e.g., on websites, mailers, flyers, product literature, etc.) The instructions for use may include a reference on how to use the unit dose measuring device to properly deliver a unit dose or fraction thereof. The instructions may additionally or alternatively include a reference to common unit dose measuring devices, such as cups, scoops, syringes, droppers, spoons, spatulas, ladles, and the like, not provided with the bulk container (e.g., in case the provided unit dose measuring device is lost or misplaced). In such case, a kit may be constructed by the end user when following instructions provided on or with the bulk container, or otherwise provided by the seller regarding the product and how to properly deliver a unit dose of composition, or fraction thereof.

### II. Nutritional Supplement Compositions

In some embodiments, the nutritional supplement compositions disclosed herein comprise most or all of the following components in relative order of prominence: cacao, collagen protein, cinnamon powder, vanilla powder, reishi mushroom powder or extract, chaga mushroom powder or extract, turkey tail mushroom powder or extract, lion's mane mushroom powder or extract, cordyceps fungus powder or extract (collectively “mushroom/fungus components”), and natural salt.

The nutritional supplement composition can be provided in a dosage form that provides the various components in the amounts specified, including the serving size amounts. The concentrations of the various components are typically in relation to the nutritional supplement composition before it is added to a liquid or solid to make an orally ingestible drink or food product. The orally ingestible drink or food product can be a dosage form that provides the desired or specified amounts of each component, but the concentrations of the various components will be reduced in proportion to the relative amounts of the composition and the liquid or solid to which the nutritional supplement composition is added or blended.

### A. Cacao

Cacao is an important component of the nutritional supplement compositions and is preferably the most prominent component in the compositions. In addition to including high amounts of theobromine and relatively minor amounts of caffeine, cacao contains other important nutrients, including flavonoids, iron, magnesium, copper, potassium, selenium, zinc, and phosphorus. The cacao component provides several benefits, including providing antioxidant properties, increasing high density lipoprotein (HDL) production, increasing mental and physical energy, improving skin health, and improving overall brain function and cognition. The cacao can also potentiate and synergistically interact with the other components to enhance their benefits, including by increasing the pharmacokinetic distribution of beneficial substances within the other components in the nutritional supplement compositions and nutritional products made therefrom.

Cacao contains a high ratio of theobromine to caffeine and therefore improves cognitive function and provides mental clarify, focus, and alertness without the jitters, heart palpitations, and other negative effects caused by highly caffeinated energy drinks and other products. While consuming high amount of caffeine can increase blood pressure, theobromine can lower blood pressure. When consumed together the net effect of theobromine and caffeine can be similar or lower blood pressure depending on the ratio and amount consumed. Different kinds of cacao and processing methods can yield cacao powder and other forms of cacao with varying ratios of theobromine to caffeine. Depending on the desired ratio, it may be desirable to add an additional quantity of theobromine and/or caffeine to yield the desired ratio. The vasodilation and energy-enhancing properties of theobromine and caffeine can enhance the beneficial effects of the other components in the nutritional supplement composition.

In some embodiments, the ratio of theobromine to caffeine in the nutritional supplement composition can be in a range of about 3:1 to about 15:1, preferably in a range of about 4:1 to about 12:1, more preferably in a range of about 5:1 to about 10:1, by weight. By way of further example, the weight ratio of theobromine to caffeine can be about 3:1, 4:1, 5:1, 6:1, 7:1, 8:1, 9:1, 10:1, 11:1, 12:1, 13:1, 14:1, or 15:1, or within a range defined by any two of the foregoing ratios.

In some embodiments, the total quantity of cacao per serving of the nutritional supplement composition or product made therefrom can be in a range of about 2000 mg to about 8000 mg, preferably in a range of about 2500 mg to about 7000 mg, more preferably in a range of about 3000 mg to about 6000 mg, and most preferably in a range of about 3250 mg to about 5500 mg, per serving. By way of further example, the amount of cacao per serving can be about 2000 mg, 2500 mg, 3000 mg, 3500 mg, 3700 mg, 3900 mg, 4100 mg, 4300 mg, 4500 mg, 4750 mg, 4900 mg, 5200 mg, 5500 mg, 6000 mg, 6500 mg, 7000 mg, 7500 mg, 8000 mg, or 8500 mg, or within a range defined by any two of the foregoing amounts.

In some embodiments, the total quantity of theobromine per serving of the nutritional supplement composition or product made therefrom can be in a range of about 30 mg to about 125 mg, preferably in a range of about 35 mg to about 100 mg, more preferably in a range of about 37.5 mg to about 85 mg, and most preferably in a range of about 40 mg to about 70 mg, per serving. By way of further example, the amount of theobromine per serving can be about 25 mg, 30 mg, 35 mg, 40 mg, 45 mg, 50 mg, 55 mg, 60 mg, 65 mg, 70 mg, 75 mg, 80 mg, 90 mg, 100 mg, 110 mg, 120 mg, 130 mg, 140 mg, or 150 mg, or within a range defined by any two of the foregoing amounts.

In some embodiments, the total quantity of caffeine per serving of the nutritional supplement composition or product made therefrom can be in a range of about 3 mg to about 25 mg, preferably in a range of about 4 mg to about 20 mg, more preferably in a range of about 5 mg to about 15 mg, and most preferably in a range of about 6 mg to about 12 mg, per serving. By way of further example, the amount of caffeine per serving can be about 2 mg, 3 mg, 3 mg, 4 mg, 5 mg, 6 mg, 7 mg, 7.5 mg, 8 mg, 8.5 mg, 9 mg, 9.5 mg. 10 mg, 10.5 mg, 11 mg, 12 mg, 13 mg, 15 mg, 17.5 mg, 20 mg, 22.5 mg, 25 mg, 27.5 mg, or 30 mg, or within a range defined by any two of the foregoing amounts.

Cacao can include other methylxanthines in addition to theobromine and caffeine, such as theophylline. While the amount of theophylline is minor, some cacao sources can have higher amounts of theophylline. Theophylline can have stimulant and mood enhancing effect similar to theobromine and caffeine. The amount of theophylline in raw paste cacao is about 1/25^{th} the amount of caffeine and is therefore not present in high amounts in processed cacao. Nonetheless, theophylline and other methylxanthines are often used for their performance-enhancing effects in sports, as these drugs increase alertness, bronchodilation, and increase the rate and force of heart contraction.

In order to provide its desired benefits and also act synergistically with the other components in the nutritional supplement composition, the cacao component can be included in a concentration a range from about 15% to about 60% by weight, preferably in a range of about 20% to about 50% by weight, more preferably in a range of about 25% to about 45% by weight, and most preferably in a range of about 30% to about 40% by weight, of the nutritional supplement composition. By way of further example, the concentration of cacao in the nutritional supplement composition can be about 5%, 10%, 15%, 20%, 25%, 30%, 35%, 40%, 45%, 50%, 55%, 60%, or 65%, or within a range defined by any two of the foregoing percentages. In preferred embodiments, the foregoing concentrations are in relation to the nutritional supplement composition before it is added to a liquid or solid to make an orally ingestible product.

### B. Collagen Protein

The inclusion of relative high amounts of collagen protein, preferably beef collagen, has also been found to provide substantial benefits when included with the other components in the nutritional supplement composition. For example, collagen protein is the major protein component of soft joint tissues, including cartilage. When combined with the other components, collagen protein has been found to promote healthier joints and to relieve joint point, including pain associated with arthritis. Another benefit of collagen protein, one that has not been readily recognized, is its positive affect on mood and mental clarity, particularly when combined with cacao and the other components. Other benefits include bone loss prevention, increased skin and joint elasticity, anti-inflammatory, anti-aging, and healthy bile production. The collagen protein also appears to maintain good dispersion the other components when the nutritional supplement composition is mixed into water or other liquid, as the other components do not readily sink to the bottom of the cup over time as occurs with coffee substitutes that include mushrooms, cacao, etc.

Collagen protein is the main structural protein in the extracellular matrix of various connective tissues in beef. As the main component of connective tissue, it is the most abundant protein in mammals. 25% to 35% of mammalian protein content is collagen protein. Amino acids are bound together to form a triple helix of elongated fibril known as a collagen helix. The collagen helix is mostly found in connective tissue such as cartilage, bones, tendons, ligaments, and skin.

Beef (or bovine) collagen protein consists of Type I and Type III collagen. Most collagens comprises the amino acid sequences glycine-proline-X and glycine-X-hydroxyproline, where X can be any amino acid other than glycine, proline, and hydroxyproline. This means that glycine constitutes approximately ⅓ of the number of amino acids in collagen protein, although not exactly 33% by weight since different amino acids have different molecular weights, and not exactly 33% by number as shown below. The abundance of the various amino acids in mammalian skin (expressed as residues/1000), which approximates their preponderance in beef collagen generally, is reportedly as follows:

| | |
|---|---|
| Glycine | 329 |
| Proline | 126 |
| Alanine | 109 |
| Hydroxyproline \grave{ } | 95 |
| Glutamic acid \grave{ } | 74 |
| Arginine | 49 |
| Aspartic acid | 47 |
| Serine | 36 |
| Lysine | 29 |
| Leucine | 24 |
| Valine | 22 |
| Threonine | 19 |
| Phenylalanine | 13 |
| Isoleucine | 11 |
| Methionine | 6 |
| Hydroxylysine | 6 |
| Histidine | 5 |
| Tyrosine | 3 |
| Cysteine | 1 |
| Tryptophan | 0 |

Glycine in particular, has been found to have beneficial effects on mood and neurological function, and appears to act synergistically to improve a sense of calm focus when combined with the mind and mood enhancing effects of cacao and mushrooms in the composition. Because glycine is the most abundant amino acid in beef collagen, constituting about ⅓ of all amino acid residues therein, beef collagen is a particularly beneficial source of glycine in a natural form. In addition, glycine is a building block of collagen in the body, along with proline and hydroxyproline, the other two main amino acids, and the “minor” amino acids. Thus, administering collagen protein can help the body rebuild soft tissues. This can have the benefit of reducing pain in joints, tendons, and ligaments, improving skin structure, and preventing bone loss (because bone contains collagen that binds the calcium-based minerals together).

In some embodiments, the total quantity of collagen protein per serving of the nutritional supplement composition or product made therefrom can be in a range of about 2000 mg to about 7500 mg, preferably in a range of about 2500 mg to about 6000 mg, more preferably in a range of about 2750 mg to about 5000 mg, and most preferably in a range of about 3000 mg to about 4500 mg, per serving. By way of further example, the amount of collagen protein per serving can be about 1500 mg, 1750 mg, 2000 mg, 2500 mg, 2750 mg, 3000 mg, 3250 mg, 3500 mg, 3750 mg, 4000 mg, 4250 mg, 4500 mg, 4750 mg, 5000 mg, 5500 mg, 6000 mg, 6500 mg, 7000 mg, 7500 mg, or 8000 mg, or within a range defined by any two of the foregoing amounts.

In some embodiments, the total quantity of glycine per serving of the nutritional supplement composition or product made therefrom can be in a range of about 660 mg to about 2500 mg, preferably in a range of about 830 mg to about 2000 mg, more preferably in a range of about 910 mg to about 1660 mg, and most preferably in a range of about 1000 mg to about 1500 mg, per serving. By way of further example, the amount of glycine per serving can be about 600 mg, 650 mg, 700 mg, 800 mg, 900 mg, 1000 mg, 1090 mg, 1200 mg, 1280 mg, 1360 mg, 1450 mg, 1500 mg, 1600 mg, 1700 mg, 1800 mg, 1900 mg, 2000 mg, 2250 mg, 2500 mg, or 2750 mg or within a range defined by any two of the foregoing amounts.

In order to provide its desired benefits and also act synergistically with the other components in the nutritional supplement composition, the collagen protein component can be included in a concentration a range from about 12.5% to about 50% by weight, preferably in a range of about 15% to about 45% by weight, more preferably in a range of about 20% to about 40% by weight, and most preferably in a range of about 25% to about 35% by weight, of the nutritional supplement composition. By way of further example, the concentration of collagen protein can be about 10%, 12%, 14%, 16%, 18%, 20%, 22.5%, 25%, 27.5%, 30%, 32.5%, 35%, 37.5%, 40%, 42.5% 45%, 47.5%, or 50%, or within a range defined by any two of the foregoing percentages.

### C. Mushroom/Fungus Components

In some embodiments, the mushroom/fungus components are in the form of powder that is dispersible in a liquid, such as warm to hot water, or provided in the form of a liquid dispersion. The mushroom/fungus components comprise at least two, preferably at least three, more preferably at least four, and most preferably all five, of the following components, which can be in the form of extracts, powders, or other forms: reishi mushroom, chaga fungus, turkey tail mushroom, lion's mane mushroom, and cordyceps fungus. The mushroom/fungus components can be in the form of a powder, such as ground fruiting bodies and/or mycelium, and preferably extracts made therefrom.

Mushroom and fungus extracts can be made from raw mushroom materials by exposing them to a solvent, such as water, alcohol, or mixture thereof, to draw out and isolate the beneficial or desired compounds. By extracting mushrooms and fungi, the active ingredients are isolated and concentrated, releasing their beneficial bioactive properties and increasing their bioavailability. Mushroom/fungus extracts can come in different forms, such as powders, tinctures, and dual liquid extracts.

The mushroom/fungus components can be included in desired ratios, such as 1:3, 1:2, 1:1, 2:1, or 3:1 from one mushroom/fungus component to the next. In some embodiments, the ratio of any two mushroom/fungus components in the composition can be in a range of about 1:3 to about 3:1, or about 1:2 to about 2:1, or about 1:1.5 to about 1.5:1, or about 1:1.25 to about 1.25:1, or about 1:1.1 to about 1.1:1, or about 1:1. According to one embodiment, two, three, four, or five mushroom/fungus components are include at approximately equal ratios, i.e., about 1:1 for any two mushroom/fungus components in the composition.

In some embodiments, the total quantity of all mushroom/fungus components per serving of the nutritional supplement composition or product made therefrom can be in a range of about 1000 mg to about 3500 mg, preferably in a range of about 1200 mg to about 3000 mg, more preferably in a range of about 1350 mg to about 2750 mg, and most preferably in a range of about 1500 mg to about 2500 mg, per serving. By way of further example, the total quantity of all mushroom/fungus components per serving can be about 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2250 mg, 2500 mg, 2750 mg, 3000, or 3500 mg, or within a range defined by any two of the foregoing amounts.

In order to provide their desired benefits and also act synergistically with the other components in the nutritional supplement composition or product made therefrom, the total mushroom/fungus components can be included in a concentration a range from about 8% to about 25% by weight, preferably in a range of about 10% to about 22% by weight, more preferably in a range of about 12% to about 19% by weight, and most preferably in a range of about 14% to about 17.5% by weight, of the nutritional supplement composition. By way of further example, the concentration of total mushroom/fungus components can be about 5%, 6%, 7%, 8%, 9%, 10%, 11%, 13%, 15%, 16%, 17.5%, 19%, 21%, 23%, 25%, or 27.5%, or within a range defined by any two of the foregoing percentages.

### 1. Reishi Mushroom

The reishi mushroom component can provide various benefits, including anti-aging effects, detoxification, neuroprotection, anti-inflammatory effects, boosts immunity, regulates blood pressure, and increases HDL.

In some embodiments, the quantity of the reishi mushroom component per serving of the nutritional supplement composition or product made therefrom can be in a range of about 200 mg to about 750 mg, preferably in a range of about 250 mg to about 600 mg, more preferably in a range of about 275 mg to about 500 mg, and most preferably in a range of about 300 mg to about 475 mg, per serving. By way of further example, the quantity of the reishi mushroom component per serving can be about 200 mg, 225 mg, 250 mg, 275 mg, 300 mg, 325 mg, 350 mg, 375 mg, 400 mg, 425 mg, 450 mg, 475 mg, 500 mg, 550 mg, 600 mg, 650 mg, 700 mg, 750 mg, or 800 mg, or within a range defined by any two of the foregoing amounts. In some embodiments, the total amount of the reishi mushroom component is preferably less than about 600 mg, or less than about 550 mg, or less than about 500 mg, or less than about 450 mg, or less than about 400 mg, or less than about 375 mg, or less than about 350 mg, per serving.

In order to provide its desired benefits and also act synergistically with the other components in the nutritional supplement composition or product made therefrom composition, the reishi mushroom component can be included in a concentration a range from about 1.5% to about 4.5% by weight, preferably in a range of about 2.25% to about 4% by weight, more preferably in a range of about 2.5% to about 3.75% by weight, and most preferably in a range of about 2.75% to about 3.5% by weight, of the nutritional supplement composition. By way of further example, the concentration of the reishi mushroom component can be about 1.4%, 1.6%, 1.8%, 2%, 2.2%, 2.4%, 2.6%, 2.8%, 3%, 3.1%, 3.2%, 3.4%, 3.6%, 3.8%, 4%, 4.25%, or 4.5%, or within a range defined by any two of the foregoing percentages.

### 2. Chaga Fungus

The chaga fungus component can provide various benefits, including high in fiber, antioxidant, providing anti-inflammatory activity, regulates blood sugar, increases HDL, high in vitamin E, energy booster, and antiviral.

In some embodiments, the quantity of the chaga fungus component per serving of the nutritional supplement composition or product made therefrom can be in a range of about 200 mg to about 750 mg, preferably in a range of about 250 mg to about 600 mg, more preferably in a range of about 275 mg to about 500 mg, and most preferably in a range of about 300 mg to about 475 mg, per serving. By way of further example, the quantity of the chaga fungus component per serving can be about 200 mg, 225 mg, 250 mg, 275 mg, 300 mg, 325 mg, 350 mg, 375 mg, 400 mg, 425 mg, 450 mg, 475 mg, 500 mg, 550 mg, 600 mg, 650 mg, 700 mg, 750 mg, or 800 mg, or within a range defined by any two of the foregoing amounts. In some embodiments, the total amount of the chaga fungus component is preferably less than about 600 mg, or less than about 550 mg, or less than about 500 mg, or less than about 450 mg, or less than about 400 mg, or less than about 375 mg, or less than about 350 mg, per serving.

In order to provide its desired benefits and also act synergistically with the other components in the nutritional supplement composition or product made therefrom, the chaga fungus component can be included in a concentration a range from about 1.5% to about 4.5% by weight, preferably in a range of about 2.25% to about 4% by weight, more preferably in a range of about 2.5% to about 3.75% by weight, and most preferably in a range of about 2.75% to about 3.5% by weight, of the nutritional supplement composition. By way of further example, the concentration of the chaga fungus component can be about 1.4%, 1.6%, 1.8%, 2%, 2.2%, 2.4%, 2.6%, 2.8%, 3%, 3.1%, 3.2%, 3.4%, 3.6%, 3.8%, 4%, 4.25%, or 4.5%, or within a range defined by any two of the foregoing percentages.

### 3. Turkey Tail Mushroom

The turkey tail mushroom component can provide various benefits, including immunity booster, regulates blood sugar, anti-inflammatory activity, and being high in potassium, riboflavin, niacin, phosphorus, and copper.

In some embodiments, the quantity of the turkey tail mushroom component per serving of the nutritional supplement composition or product made therefrom can be in a range of about 200 mg to about 750 mg, preferably in a range of about 250 mg to about 600 mg, more preferably in a range of about 275 mg to about 500 mg, and most preferably in a range of about 300 mg to about 475 mg, per serving. By way of further example, the quantity of the turkey tail mushroom component per serving can be about 200 mg, 225 mg, 250 mg, 275 mg, 300 mg, 325 mg, 350 mg, 375 mg, 400 mg, 425 mg, 450 mg, 475 mg, 500 mg, 550 mg, 600 mg, 650 mg, 700 mg, 750 mg, or 800 mg, or within a range defined by any two of the foregoing amounts. In some embodiments, the total amount of the turkey tail mushroom component is preferably less than about 600 mg, or less than about 550 mg, or less than about 500 mg, or less than about 450 mg, or less than about 400 mg, or less than about 375 mg, or less than about 350 mg, per serving.

In order to provide its desired benefits and also act synergistically with the other components in the nutritional supplement composition or product made therefrom, the turkey tail mushroom component can be included in a concentration a range from about 1.5% to about 4.5% by weight, preferably in a range of about 2.25% to about 4% by weight, more preferably in a range of about 2.5% to about 3.75% by weight, and most preferably in a range of about 2.75% to about 3.5% by weight, of the nutritional supplement composition. By way of further example, the concentration of the turkey tail mushroom component can be about 1.4%, 1.6%, 1.8%, 2%, 2.2%, 2.4%, 2.6%, 2.8%, 3%, 3.1%, 3.2%, 3.4%, 3.6%, 3.8%, 4%, 4.25%, or 4.5%, or within a range defined by any two of the foregoing percentages.

### 4. Lion's Mane Mushroom

The lion's mane mushroom component can provide various benefits, including being an adaptogen, anti-inflammatory activity, antioxidant, probiotic, regulates blood sugar, and regulates blood pressure. Some research supports positive effects on depression and anxiety.

In some embodiments, the quantity of the lion's mane mushroom component per serving of the nutritional supplement composition or product made therefrom can be in a range of about 200 mg to about 750 mg, preferably in a range of about 250 mg to about 600 mg, more preferably in a range of about 275 mg to about 500 mg, and most preferably in a range of about 300 mg to about 475 mg, per serving. By way of further example, the quantity of the lion's mane mushroom component per serving can be about 200 mg, 225 mg, 250 mg, 275 mg, 300 mg, 325 mg, 350 mg, 375 mg, 400 mg, 425 mg, 450 mg, 475 mg, 500 mg, 550 mg, 600 mg, 650 mg, 700 mg, 750 mg, or 800 mg, or within a range defined by any two of the foregoing amounts. In some embodiments, the total amount of the lion's mane mushroom component is preferably less than about 600 mg, or less than about 550 mg, or less than about 500 mg, or less than about 450 mg, or less than about 400 mg, or less than about 375 mg, or less than about 350 mg, per serving.

In order to provide its desired benefits and also act synergistically with the other components in the nutritional supplement composition or product made therefrom, the lion's mane mushroom component can be included in a concentration a range from about 1.5% to about 4.5% by weight, preferably in a range of about 2.25% to about 4% by weight, more preferably in a range of about 2.5% to about 3.75% by weight, and most preferably in a range of about 2.75% to about 3.5% by weight, of the nutritional supplement composition. By way of further example, the concentration of the lion's mane mushroom component can be about 1.4%, 1.6%, 1.8%, 2%, 2.2%, 2.4%, 2.6%, 2.8%, 3%, 3.1%, 3.2%, 3.4%, 3.6%, 3.8%, 4%, 4.25%, or 4.5%, or within a range defined by any two of the foregoing percentages.

### 5. Cordyceps Fungus

The cordyceps fungus component can provide various benefits, including anti-inflammatory activity, antioxidant, energy booster, regulates blood pressure, regulates blood sugar, anti-aging, and hormone regulator.

In some embodiments, the quantity of the cordyceps fungus component per serving of the nutritional supplement composition or product made therefrom can be in a range of about 200 mg to about 750 mg, preferably in a range of about 250 mg to about 600 mg, more preferably in a range of about 275 mg to about 500 mg, and most preferably in a range of about 300 mg to about 475 mg, per serving. By way of further example, the quantity of the cordyceps fungus component per serving can be about 200 mg, 225 mg, 250 mg, 275 mg, 300 mg, 325 mg, 350 mg, 375 mg, 400 mg, 425 mg, 450 mg, 475 mg, 500 mg, 550 mg, 600 mg, 650 mg, 700 mg, 750 mg, or 800 mg, or within a range defined by any two of the foregoing amounts. In some embodiments, the total amount of the cordyceps fungus component is preferably less than about 600 mg, or less than about 550 mg, or less than about 500 mg, or less than about 450 mg, or less than about 400 mg, or less than about 375 mg, or less than about 350 mg, per serving.

In order to provide its desired benefits and also act synergistically with the other components in the nutritional supplement composition or product made therefrom, the cordyceps fungus component can be included in a concentration a range from about 1.5% to about 4.5% by weight, preferably in a range of about 2.25% to about 4% by weight, more preferably in a range of about 2.5% to about 3.75% by weight, and most preferably in a range of about 2.75% to about 3.5% by weight, of the nutritional supplement composition. By way of further example, the concentration of the cordyceps fungus component can be about 1.4%, 1.6%, 1.8%, 2%, 2.2%, 2.4%, 2.6%, 2.8%, 3%, 3.1%, 3.2%, 3.4%, 3.6%, 3.8%, 4%, 4.25%, or 4.5%, or within a range defined by any two of the foregoing percentages.

### D. Cinnamon

The cinnamon component, preferably Ceylon cinnamon, can provide various benefits, including antioxidant, high in beta-carotene (which is able to convert in the body to vitamin A), anti-inflammatory activity, regulates blood sugar, and is rich in manganese, vitamin k, potassium, zinc and magnesium.

In some embodiments, the quantity of cinnamon per serving of the nutritional supplement composition or product made therefrom can be in a range of about 600 mg to about 2000 mg, preferably in a range of about 700 mg to about 1500 mg, more preferably in a range of about 750 mg to about 1250 mg, and most preferably in a range of about 800 mg to about 1150 mg, per serving. By way of further example, the quantity of cinnamon per serving can be about 500 mg, 600 mg, 700 mg, 800 mg, 825 mg, 900 mg, 1000 mg, 1050 mg, 1100 mg, 1200 mg, 1350 mg, 1500 mg, 1750 mg, 2000 mg, or 2250 mg, or within a range defined by any two of the foregoing amounts.

In order to provide its desired benefits and also act synergistically with the other components in the nutritional supplement composition or product made therefrom, the quantity of cinnamon can be included in a concentration a range from about 5% to about 15% by weight, preferably in a range of about 6% to about 12% by weight, more preferably in a range of about 6.5% to about 10% by weight, and most preferably in a range of about 7% to about 9% by weight, of the nutritional supplement composition. By way of further example, the concentration of cinnamon can be about 4%, 5%, 5.5%, 6%, 6.5%, 7%, 7.5%, 8%, 8.5%, 9%, 9.5%, 10%, 12.5%, 15%, 17.5%, or 20%, or within a range defined by any two of the foregoing percentages.

### E. Vanilla

The vanilla component can provide various benefits, including antioxidant, anti-inflammatory activity, and neuroprotection. The vanilla component can be provided as a concentrated powder or in a liquid solution or suspension.

In some embodiments, the quantity of vanilla per serving of the nutritional supplement composition or product made therefrom can be in a range of about 500 mg to about 1750 mg, preferably in a range of about 600 mg to about 1250 mg, more preferably in a range of about 650 mg to about 1100 mg, and most preferably in a range of about 700 mg to about 1000 mg, per serving. By way of further example, the quantity of vanilla per serving can be about 400 mg, 500 mg, 600 mg, 650 mg, 700 mg, 725 mg, 750 mg, 800 mg, 850 mg, 900 mg, 950 mg, 1000 mg, 1100 mg, 1250 mg, 1400 mg, 1600 mg, 1750 mg, 1750 mg, or 2000 mg, or within a range defined by any two of the foregoing amounts.

In order to provide its desired benefits and also act synergistically with the other components in the nutritional supplement composition or product made therefrom, the quantity of vanilla can be included in a concentration a range from about 4% to about 15% by weight, preferably in a range of about 5.5% to about 12% by weight, more preferably in a range of about 6.5% to about 10% by weight, and most preferably in a range of about 6% to about 8% by weight, of the nutritional supplement composition. By way of further example, the concentration of vanilla can be about 3%, 3.5%, 4%, 4.5%, 5%, 5.5%, 6%, 6.5%, 7%, 7.5%, 8%, 9%, 10%, 12%, 15%, or 18%, or within a range defined by any two of the foregoing percentages.

### F. Salt

The salt component is preferably a natural salt (not refined) salt, such as “real salt” provided by Redmond Life, headquartered in Heber City, Utah, with the salt being mined in Redmond, Utah. Natural salts, such as Redmond salt, Himalayan salt, and the like, can provide various benefits, including providing electrolytes, promoting water retention, and offsetting the diuretic effects of theobromine and caffeine contained in the cacao component. Natural salt, such as Redmond salt, regulates blood sugar, electrolyzes water molecules, maintains pH, lowers adrenaline spikes, improves melatonin production, encourages metabolic health, supports thyroid function, and supports iodine utilization.

The reported mineral content of Redmond real salt from the Redmond salt web page is provided in Table 1 below.

TABLE 1

| Element | ≈ < % | Element | ≈ < % | Element | ≈ < % |
|---|---|---|---|---|---|
| Chloride | 61.0 | Gallium | 0.000100 | Rhodium [symbol] | 0.0000003 |
| Sodium | 38.0 | Germanium [symbol] | 0.000060 | Rubidium | 0.000025 |
| Calcium | 0.500000 | Gold [symbol] | 0.000080 | Ruthenium [symbol] | 0.000010 |
| Magnesium | 0.120000 | Hafnium [symbol] | 0.000020 | Samarium | 0.000020 |
| Potassium | 0.100000 | Holmium [symbol] | 0.0000004 | Scandium | 0.000040 |
| Aluminum | 0.050000 | Indium [symbol] | 0.000080 | Selenium | 0.000100 |
| Antimony [symbol] | 0.000700 | Iodine | 0.003000 | Silicon | 0.050000 |
| Arsenic [symbol] | 0.000008 | Iridium [symbol] | <0.0000003 | Silver [symbol] | 0.000200 |
| Barium | 0.005000 | Iron | 0.030000 | Strontium | 0.003000 |
| Beryllium [symbol] | 0.000100 | Lanthanum [symbol] | 0.000020 | Sulfur | 0.200000 |
| Bismuth [symbol] | 0.000500 | Lead [symbol] | 0.000020 | Tantaium [symbol] | 0.0000003 |
| Boron | 0.000200 | Lithium | 0.000100 | Tellurium [symbol] | 0.000900 |
| Bromine [symbol] | 0.050000 | Lutetium | 0.000009 | Terbium [symbol] | 0.0000005 |
| Cadmium | 0.000002 | Manganese | 0.002000 | Thallium [symbol] | 0.000500 |
| Carbon | 0.050000 | Mercury [symbol] | 0.000002 | Thorium [symbol] | 0.000500 |
| Cerium | 0.000050 | Molybdenum [symbol] | 0.000070 | Thulium [symbol] | 0.0000002 |
| Cesium | 0.000005 | Neodymium [symbol] | 0.000060 | Tin [symbol] | 0.000008 |
| Chromium [symbol] | 0.000020 | Nickel [symbol] | 0.000009 | Titanium | 0.000500 |
| Cobalt | 0.000006 | Niobium [symbol] | 0.000020 | Tungsten [symbol] | 0.000800 |
| Copper [symbol] | 0.000200 | Osmium [symbol] | <0.0000003 | Uranium [symbol] | <0.001 |
| Dysprosium | 0.000002 | Palladium [symbol] | 0.000003 | Vanadium [symbol] | 0.000020 |
| Erbium | 0.000002 | Phosphorous | 0.003000 | Ytterbium | 0.0000009 |
| Europium [symbol] | 0.000002 | Platinum [symbol] | <0.001 | Yttrium [symbol] | 0.000009 |
| Fluoride [symbol] | 0.002000 | Praseodymium | 0.000003 | Zinc | 0.000200 |
| Gadolinium | 0.000002 | Rhenium [symbol] | <0.001 | Zirconium | 0.000300 |

[symbol]

indicates data missing or illegible when filed

In some embodiments, the quantity of the natural salt component per serving of the nutritional supplement composition or product made therefrom can be in a range of about 200 mg to about 600 mg, preferably in a range of about 250 mg to about 500 mg, more preferably in a range of about 275 mg to about 450 mg, and most preferably in a range of about 300 mg to about 425 mg, per serving. By way of further example, the quantity of the natural salt component per serving can be about 200 mg, 225 mg, 250 mg, 275 mg, 300 mg, 325 mg, 350 mg, 375 mg, 400 mg, 425 mg, 450 mg, 475 mg, 500 mg, 550 mg, 600 mg, 650 mg, or 700 mg, or within a range defined by any two of the foregoing amounts.

In order to provide its desired benefits and also act synergistically with the other components in the nutritional supplement composition or product made therefrom, the natural salt component can be included in a concentration a range from about 2% to about 4.5% by weight, preferably in a range of about 2.25% to about 4% by weight, more preferably in a range of about 2.5% to about 3.5% by weight, and most preferably in a range of about 2.75% to about 3.25% by weight, of the composition. By way of further example, the concentration of the natural salt component can be about 1.5%, 1.7%, 1.9%, 2.1%, 2.3%, 2.5%, 2.7%, 2.9%, 3%, 3.1%, 3.3%, 3.5%, 3.75%, 4%, 4.25%, 4.5%, 4.75%, or 5%, or within a range defined by any two of the foregoing percentages.

### III. Delivery Methods and Dosage Forms

The nutritional supplement composition may be consumed or administered through any acceptable administration route. In a first embodiments, the composition is formulated as a powder that can be added to a liquid, such as water, milk, or juice, for oral consumption as a beverage. When making the beverage, the user can add other components for taste or nutrition, such as a sweetener (e.g., honey, agave syrup, and coconut sugar), and coconut oil, which is a source of medium chain triglycerides (MCT). The nutritional supplement powder mixes well when using warm or hot water, milk, or other liquid. The composition in powder form can be provided in a bulk container along with a measuring device that allows the user to place a premeasured dose of the powder into a cup for mixing with water, milk, or other liquid. The composition can be administered once daily or multiple times daily (e.g., 2-5 times).

In a third embodiment, the nutritional supplement composition may be in the form of a ready to use beverage pre-packaged in a container. When formulated as a beverage, the composition may be provided in a can, bottle, flask, or other such container configured to hold a desired number of unit doses of the composition. A container may hold, for example, about 2 ounces to about 40 ounces of the composition, more typically about 2.5 ounces to 24 ounces, or about 3 ounces to about 12 ounces. The separate components of the nutritional supplement composition may be diluted or concentrated accordingly so as to provide desired levels of each component in each corresponding unit dose of the container. For example, a smaller supplement “shot” may be more concentrated than a larger and more dilute beverage.

### IV. Examples

The following is a description of an exemplary nutritional supplement composition that has been made and found useful for enhancing mental focus, reducing pain, including pain from arthritis, and improving gastrointestinal (GI) tract function (e.g., bowel function).

### Example 1A

| Component | Weight (oz or gram) | Weight % |
|---|---|---|
| Reishi | 180 | 2.68% |
| Chaga | 180 | 2.68% |
| Turkey tail | 180 | 2.68% |
| Lions Mane | 180 | 2.68% |
| Cordyceps | 180 | 2.68% |
| Cacao | 2043 | 30.37% |
| Cinnamon | 907 | 13.48% |
| Vanilla powder | 907 | 13.48% |
| Salt | 170 | 2.53% |
| Collagen protein | 1800 | 26.76% |
| Total | 6727 | 100.00% |

The nutritional supplement composition was in the form a powder that was added to warm or hot water or milk to form a beverage. In some cases, the beverage was augmented with a sweetener, such as honey. In other cases, the beverage was augmented with a source of medium chain fatty acids (e.g., coconut oil).

### Example 1B

The nutritional supplement composition can be administered in any desired quantity. In this example, a unit dose of the composition can be about 2 tablespoons (29.57 mL) or about 1.5 tablespoon (22.18 mL). The amounts of the various components in the nutritional supplement composition for a unit does of 2 tablespoons or 1.5 tablespoon, respectively, were as follows:

| Component | Amount in 2 Tbsp | | Amount in 1.5 Tbsp | |
|---|---|---|---|---|
| Reishi | 375 | mg | 281 | mg |
| Chaga | 375 | mg | 281 | mg |
| Turkey tail | 375 | mg | 281 | mg |
| Lions Mane | 375 | mg | 281 | mg |
| Cordyceps | 375 | mg | 281 | mg |
| Cacao | 4252 | mg | 3189 | mg |
| Cinnamon | 1888 | mg | 1416 | mg |
| Vanilla powder | 1888 | mg | 1416 | mg |
| Salt | 354 | mg | 265 | mg |
| Collagen protein | 3746 | mg | 2810 | mg |
| Total | 14,000 | mg | 10,500 | mg |

The nutritional supplement composition has been found to provide improved mental clarity, including treating the effects of attention deficit disorder (ADD), without the jitteriness associated with typical highly caffeinated beverages. The composition has also been found to provide additional unexpected health benefits including, but not limited to, boosting energy, generally reducing pain, including reducing pain associated with arthritis, increasing youthfulness, improving gastrointestinal function, including improving bowel function, improving immunity, increasing rate of wound healing, improving skin health and elasticity, increasing HDL production, providing anti-inflammatory effects, providing anti-oxidant activity, providing anti-viral activity, improves cognitive function, providing probiotics, improving mood, reducing depression and anxiety, providing neuroprotection, boosting immunity, regulating blood pressure, regulating blood sugar, anti-aging, hormone regulation, reducing adrenaline spikes, maintaining pH, improving melatonin production, improving metabolism, supporting thyroid function, reducing the desire to consume sugar and other high glycemic index carbohydrates.

The composition of Examples 1A and 1B was reformulated to make another exemplary nutritional supplement composition that has been made and found useful for enhancing mental focus, reducing pain, including pain from arthritis, and improving gastrointestinal (GI) tract function (e.g., bowel function).

### Example 2A

| Component | Weight (oz or gram) | Weight % |
|---|---|---|
| Reishi | 180 | 3.12% |
| Chaga | 180 | 3.12% |
| Turkey tail | 180 | 3.12% |
| Lions Mane | 180 | 3.12% |
| Cordyceps | 180 | 3.12% |
| Cacao | 2043 | 35.43% |
| Cinnamon | 454 | 7.87% |
| Vanilla powder | 400 | 6.94% |
| Salt | 170 | 2.95% |
| Collagen protein | 1800 | 31.21% |
| Total | 5767 | 100.00% |

The nutritional supplement composition was in the form a powder that was added to warm or hot water or milk to form a beverage. In some cases, the beverage was augmented with a sweetener, such as honey. In other cases, the beverage was augmented with a source of medium chain fatty acids (e.g., coconut oil). The percent reductions in cinnamon and vanilla reduced their relative effects, and the percent increases in the other components increased their relative effects, including increased metal focus, clarity, and joint heath provided by combination of the mushroom/fungus components, cacao, and collagen protein.

### Example 2B

The nutritional supplement composition can be administered in any desired quantity. In this example, a unit dose of the composition can be about 2 tablespoons (29.57 mL) or about 1.5 tablespoon (22.18 mL). The amounts of the various components in the nutritional supplement composition for a unit does of 2 tablespoons or 1.5 tablespoon, respectively, were as follows:

| Component | Amount in 2 Tbsp | | Amount in 1.5 Tbsp | |
|---|---|---|---|---|
| Reishi | 437 | mg | 328 | mg |
| Chaga | 437 | mg | 328 | mg |
| Turkey tail | 437 | mg | 328 | mg |
| Lions Mane | 437 | mg | 328 | mg |
| Cordyceps | 437 | mg | 328 | mg |
| Cacao | 4960 | mg | 3720 | mg |
| Cinnamon | 1102 | mg | 827 | mg |
| Vanilla powder | 971 | mg | 728 | mg |
| Salt | 413 | mg | 310 | mg |
| Collagen protein | 4370 | mg | 3277 | mg |
| Total | 14,000 | mg | 10,500 | mg |

The nutritional supplement composition has been found to provide improved mental clarity, including treating the effects of attention deficit disorder (ADD), without the jitteriness associated with typical highly caffeinated beverages. The composition has also been found to provide additional unexpected health benefits including, but not limited to, boosting energy, generally reducing pain, including reducing pain associated with arthritis, increasing youthfulness, improving gastrointestinal function, including improving bowel function, improving immunity, increasing rate of wound healing, improving skin health and elasticity, increasing HDL production, providing anti-inflammatory effects, providing anti-oxidant activity, providing anti-viral activity, improves cognitive function, providing probiotics, improving mood, reducing depression and anxiety, providing neuroprotection, boosting immunity, regulating blood pressure, regulating blood sugar, anti-aging, hormone regulation, reducing adrenaline spikes, maintaining pH, improving melatonin production, improving metabolism, supporting thyroid function, reducing the desire to consume sugar and other high glycemic index carbohydrates.

### Example 3

The nutritional supplement composition in either of Examples 1A-1B or 2A-2B is modified by incorporating the composition into a solid to make a food product that provides the same or similar nutritional benefits as when used to make a beverage.

### Example 4

A 49-year old man consumed the nutritional supplement composition of Examples 2A-2B by mixing it in warm or hot water and adding honey and coconut oil to form a good tasting beverage. The man found that the beverage improved his mental focus and clarity. The man also found that the composition was effective in reducing bodily aches and pain and increasing the rate of wound healing (i.e., he noticed that a cut sealed off in 20 hours rather than the usual 3 days, and the injury was barely noticeable after 5 days. The beverage also provided physical energy, given the man a more youthful feeling, and reduced the craving for sweets and sugar. The beverage also reduced the incidence of sickness.

### Example 5

A 47-year old woman and wife of the man in Example 4 consumed the nutritional supplement composition of Examples 2A-2B as a beverage and found that it improved her bowel function after trying many other failed remedies.

### Example 6

An older woman and the mother of the man in Example 4 consumed the nutritional supplement composition of Examples 2A-2B as a beverage and found that it greatly improved her arthritis pain, reducing it by 90%.

### Example 7

An elderly woman consumed the nutritional supplement composition of Examples 2A-2B experimentally as a beverage and found that it reduced the incidence of sickness, and she avoided getting the usual late winter sicknesses.

The claimed invention may be embodied in other specific forms without departing from its spirit or essential characteristics. The described embodiments are to be considered in all respects only as illustrative and not restrictive. The scope of the claimed invention is, therefore, indicated by the appended claims rather than by the foregoing description. All changes that come within the meaning and range of equivalency of the claims are to be embraced within their scope.

1. A nutritional supplement composition for use in providing at least one of improved mental focus and clarity, improved bowel function, or decreased pain associated with arthritis, the composition comprising:
cacao at a concentration of about 10% to about 50% by weight of the nutritional supplement composition;
collagen protein at a concentration of about 8% to about 45% by weight of the nutritional supplement composition;
a mushroom/fungus component comprising a plurality of mushroom or fungus powders or extracts selected from reishi, chaga, turkey tail, lion's mane, and cordyceps, wherein the mushroom/fungus component is at a concentration of about 5% to about 22% by weight of the nutritional supplement composition;
cinnamon;
vanilla; and
salt.

2. The nutritional supplement composition of claim 1, wherein the nutritional supplement composition includes theobromine and optionally caffeine, and wherein a weight ratio of the theobromine to the optional caffeine is at least about 5:1, or at least about 6:1, or at least about 7:1, or at least about 8:1, or at least about 9:1, or at least about 10:1.

3. The nutritional supplement composition of claim 1, wherein the nutritional supplement composition comprises cacao at a concentration of about 15% to about 60%, or about 20% to about 50%, or about 25% to about 45%, or about 30% to about 40%, by weight.

4. The nutritional supplement composition of claim 1, wherein the nutritional supplement composition is in a dosage form that provides cacao in an amount in a range about 2000 mg to about 8000 mg, or about 2500 mg to about 7000 mg, or about 3000 mg to about 6000 mg, or about 3250 mg to about 5500 mg.

5. The nutritional supplement composition of claim 1, wherein the nutritional supplement composition is in a dosage form that provides theobromine in an amount in a range of about 30 mg to about 125 mg, or in a range of about 35 mg to about 100 mg, or in a range of about 37.5 mg to about 85 mg, or in a range of about 40 mg to about 70 mg.

6. The nutritional supplement composition of claim 1, wherein the nutritional supplement composition is in a dosage form that provides caffeine in an amount in a range of about 3 mg to about 25 mg, or in a range of about 4 mg to about 20 mg, or in a range of about 5 mg to about 15 mg, or in a range of about 6 mg to about 12 mg.

7. The nutritional supplement composition of claim 1, wherein the nutritional supplement composition comprises collagen protein at a concentration of about 12.5% to about 50%, or about 15% to about 45%, or about 20% to about 40%, or about 25% to about 35%, by weight.

8. The nutritional supplement composition of claim 1, wherein the nutritional supplement composition is in a dosage form that provides collagen protein in an amount in a range about 2000 mg to about 7500 mg, or about 2500 mg to about 6000 mg, or about 2750 mg to about 5000 mg, or about 3000 mg to about 4500 mg.

9. The nutritional supplement composition of claim 1, wherein the nutritional supplement composition is in a dosage form that provides glycine in an amount in a range about 660 mg to about 2500 mg, or about 830 mg to about 2000 mg, or about 910 mg to about 1660 mg, or about 1000 mg to about 1500 mg.

10. The nutritional supplement composition of claim 1, wherein the nutritional supplement composition comprises the mushroom/fungus component at a concentration of about 8% to about 25%, or about 10% to about 22%, or about 12% to about 19%, or about 14% to about 17.5%, by weight.

11. The nutritional supplement composition of claim 1, wherein the nutritional supplement composition is in a dosage form that provides the mushroom/fungus component in an amount in a range about 1000 mg to about 3500 mg, or about 1200 mg to about 3000 mg, or about 1350 mg to about 2750 mg, or about 1500 mg to about 2500 mg.

12. The nutritional supplement composition of claim 1, wherein the nutritional supplement composition comprises at least three, at least four, or all five, of the mushroom or fungus powders or extracts selected from reishi, chaga, turkey tail, lion's mane, and cordyceps.

13. The nutritional supplement composition of claim 12, wherein the nutritional supplement composition comprises:
reishi mushroom powder or extract at a concentration of about 1% to about 4%, or about 1.5% to about 4.5%, or about 2.25% to about 4%, or about 2.5% to about 3.75%, or about 2.75% to about 3.5%, by weight; and/or
chaga fungus powder or extract at a concentration of about 1% to about 4%, or about 1.5% to about 4.5%, or about 2.25% to about 4%, or about 2.5% to about 3.75%, or about 2.75% to about 3.5%, by weight; and/or
turkey tail mushroom powder or extract at a concentration of about 1% to about 4%, or about 1.5% to about 4.5%, or about 2.25% to about 4%, or about 2.5% to about 3.75%, or about 2.75% to about 3.5%, by weight; and/or
lion's mane mushroom powder or extract at a concentration of about 1% to about 4%, or about 1.5% to about 4.5%, or about 2.25% to about 4%, or about 2.5% to about 3.75%, or about 2.75% to about 3.5%, by weight; and/or
cordyceps fungus powder or extract at a concentration of about 1% to about 4%, or about 1.5% to about 4.5%, or about 2.25% to about 4%, or about 2.5% to about 3.75%, or about 2.75% to about 3.5%, by weight.

14. The nutritional supplement composition of claim 1, wherein the nutritional supplement composition comprises cinnamon at a concentration of about 5% to about 15%, or about 6% to about 12%, or about 6.5% to about 10%, or about 7% to about 9%, by weight.

15. The nutritional supplement composition of claim 1, wherein the nutritional supplement composition comprises vanilla at a concentration of 4% to about 15%, or about 5.5% to about 12%, or about 6.5% to about 10%, or about 6% to about 8%, by weight.

16. The nutritional supplement composition of claim 1, wherein the salt comprises natural salt, and wherein the nutritional supplement composition comprises salt at a concentration of 2% to about 4.5%, or about 2.25% to about 4%, or about 2.5% to about 3.5%, or about 2.75% to about 3.25%, by weight.

17. A beverage or food composition comprising the nutritional supplement composition of claim 1.

18. A nutritional supplement composition for providing at least one of improved mental focus and clarity, improved bowel function, or decreased pain associated with arthritis, the nutritional supplement composition comprising:
cacao at a concentration of about 30% to about 40% by weight of the nutritional supplement composition;
collagen protein at a concentration of about 25% to about 35% by weight of the nutritional supplement composition;
a mushroom/fungus component comprising mushroom or fungus powders or extracts of reishi, chaga, turkey tail, lion's mane, and cordyceps, wherein the mushroom/fungus component is at a concentration of about 12% to about 19% by weight of the nutritional supplement composition;
cinnamon at a concentration of about 6.5% to about 10% by weight of the nutritional supplement composition;
vanilla at a concentration of about 6.5% to about 10% by weight of the nutritional supplement composition; and
salt at a concentration of about 2.5% to about 3.5% by weight of the nutritional supplement composition.

19. A beverage or food composition comprising the nutritional supplement composition of claim 18.

20. A nutritional supplement composition for providing at least one of improved mental focus and clarity, improved bowel function, or decreased pain associated with arthritis, the nutritional supplement composition comprising:
cacao in an amount of about 3250 mg to about 5500 mg;
collagen protein in an amount of about 3000 mg to about 4500 mg;
a mushroom/fungus component comprising mushroom or fungus powders or extracts of reishi, chaga, turkey tail, lion's mane, and cordyceps, wherein the mushroom/fungus component is in an amount of about 1500 mg to about 2500 mg;
cinnamon in an amount of about 750 mg to about 1250 mg;
vanilla in an amount of about 650 mg to about 1100 mg; and
salt in an amount of about 275 mg to about 450 mg.
